(19) United States
(12) Patent Application Publication
(10) Pub. No.: US 2020/0051705 A1
(43) Pub. Date: Feb. 13, 2020

(54) NECK SUPPORT WITH RADIATION PROTECTION

(71) Applicant: Derek T. Bernstein, Philadelphia, PA (US)

(72) Inventors: Derek T. Bernstein, Philadelphia, PA (US); Bradley K. Weiner, Houston, TX (US)

(21) Appl. No.: 16/485,817

(22) PCT Filed: Feb. 15, 2018
(86) PCT No.: PCT/US18/18379

§ 371 Date: Aug. 14, 2019

Related U.S. Application Data
(60) Provisional application No. 62/459,317, filed on Feb. 15, 2017.

(51) Int. Cl. G21F 3/00; A61B 90/60; G21F 1/08

(52) U.S. Cl. CPC G21F 3/00; A61N 2005/1094; G21F 1/08; A61B 90/60

(57) ABSTRACT

A novel radiation-protective neck support which provides cervical support and radiation protection for the neck and thyroid of a user, said support comprises (i) a soft collar portion which comprises a soft, flexible supportive material structure and is “leaded”, and (ii) a thyroid shield portion which descends from the soft collar portion and is “leaded”. The thyroid shield portion can descend as a “flap” from the soft collar portion or it can attach in other ways to achieve the protection desired for the thyroid. During surgery, the radiation-protective neck support is worn by a user, with the soft collar portion surrounding the neck of the user, thereby providing anatomical support and also providing radiation protection for the neck. With the thyroid shield portion descending as a “flap” from the soft collar portion, it will overlay the thyroid of the user and thereby provide radiation protection for the thyroid.

### REFERENCE TO PENDING PRIOR PATENT APPLICATION

This patent application claims benefit of pending prior U.S. Provisional Patent Application Ser. No. 62/459,317, filed Feb. 15, 2017 by The Methodist Hospital and Derek T. Bernstein et al. for NECK SUPPORT WITH RADIATION PROTECTION (Attorney's Docket No. METHODIST-36 PROV), which patent application is hereby incorporated herein by reference.

### FIELD OF THE INVENTION

This invention relates to medical apparatus in general, and more particularly to medical apparatus worn by medical personnel while performing medical procedures.

### BACKGROUND OF THE INVENTION

In the course of performing a surgical procedure, the surgeon may spend prolonged periods of time “hunched over” the surgical field in ergonomically-disadvantageous positions, which typically confers a significant amount of stress to the cervical and cervico-thoracic regions (i.e., the neck and the base of the neck and shoulders). As a result, neck pain is a common complaint among surgeons throughout their careers and this neck pain frequently progresses into a serious medical condition which must be addressed.

Neck-related problems in surgeons are typically treated on a case-by-case basis. Soft collars and rigid orthoses may be used to provide neck support. See, for example, the conventional soft collar 5 shown in FIG. 1. Physical therapy, NSAIDs (non-steroidal anti-inflammatory drugs), chiropractic treatment, acupuncture and/or steroid injections are other non-operative treatment options. In more serious cases, degenerative cervical spine disease with progressive neurologic compromise may occur, and surgical decompression and fusion may be required. This may result in extended time away from work and lost productivity and could even be career ending.

Furthermore, X-ray technicians have to position patients appropriately to obtain X-rays and this can, in some cases, require some “heavy lifting” which may also confer a significant amount of stress to the cervical and cervico-thoracic regions (i.e., the neck and the base of the neck and shoulders).

In addition to the foregoing, in fields such as orthopaedic surgery, radiographic visualization is typically heavily relied upon, for example, during bony procedures. Radiographic visualization may be frequently utilized in other fields as well, such as Interventional Radiology.

The effects of chronic radiation exposure have been well documented and have led to significant advances in the use of safety apparatus, including the wearing of lead vests, lead gloves, thyroid shields, eye goggles, etc. by medical personnel (e.g., surgeons, X-ray technicians, etc.).

The thyroid gland (“the thyroid”) is one of the most radiation-sensitive parts of the human body. Conventional thyroid shields are frequently used to protect the thyroid from radiation exposure. See, for example, FIG. 2 which shows a thyroid gland 10 and adjacent anatomy, and FIG. 3 which shows a conventional thyroid shield 15 being worn by a surgeon. Conventional thyroid shields do not provide effective radiation protection for other anatomical structures that are located higher in the neck, and conventional thyroid shields do not provide anatomical support for the surgeon.

Historically, soft collars have not been used for protection from radiation or cervical support to prevent injury; rather, they have primarily been used to address injuries after they have occurred, where the support function is the main object of usage. Similarly, the thyroid shield is not designed to provide anatomical or cervical support for the neck, and has not been designed to fully protect the entire neck from radiation (instead, focusing on the thyroid gland). Therefore, there is a need for a radiation-protective neck support that is radiation protective for more anatomical structures in the neck than just the thyroid gland, and that can be used to help protect against injury to the cervical and cervico-thoracic regions.

Thus there is a need for a new and improved approach for providing anatomical support for the neck of a user while simultaneously providing radiation protection for the neck and thyroid of the user.

### SUMMARY OF THE INVENTION

These and other objects of the present invention are addressed by the provision and use of a novel neck support with radiation protection.

More particularly, the present invention comprises the provision and use of a novel radiation-protective neck support which simultaneously provides both cervical support and radiation protection for the neck and thyroid of a user. The novel radiation-protective neck support generally comprises (i) a soft collar portion which comprises a soft, flexible supportive material structure (such as a supportive foam or a supportive gel pad or other supportive material with similar properties) and is “leaded” (or otherwise shielded against radiation), and (ii) a thyroid shield portion which descends from the soft collar portion and is “leaded” (or otherwise shielded against radiation). The thyroid shield portion can descend as a “flap” from the soft collar portion or it can attach in other ways to achieve the protection desired for the thyroid.

During surgery, the radiation-protective neck support is worn by the user, with the soft collar portion surrounding the neck of the user, thereby providing anatomical support to the neck of the user and also providing radiation protection for the neck of the user. With the thyroid shield portion descending as a “flap” from the soft collar portion, it will overlay the thyroid of the user and thereby provide radiation protection for the thyroid of the user.

In one form of the invention, there is provided a radiation-protective neck support comprising:

a soft collar portion comprising an anatomical support structure for the neck of a user, and radiation shielding for protection of a user's anatomy underlying the soft collar portion; and

a thyroid shield portion comprising radiation shielding for providing radiation protection to the user's anatomy underlying the thyroid shield portion, wherein the thyroid shield portion descends from the soft collar portion and is sized to cover the thyroid of a user when the soft collar portion is fitted about the neck of a user.

In another form of the invention, there is provided a method for providing neck support and radiation protection for a user, the method comprising:

providing a radiation-protective neck support, the radiation-protective neck support comprising:
a soft collar portion comprising an anatomical support structure for the neck of a user, and radiation shielding for protection of a user's anatomy underlying the soft collar portion; and
a thyroid shield portion comprising radiation shielding for providing radiation protection to the user's anatomy underlying the thyroid shield portion, wherein the thyroid shield portion descends from the soft collar portion and is sized to cover the thyroid of a user when the soft collar portion is fitted about the neck of a user; and

positioning the radiation-protective neck support on a user so that the soft collar portion is fitted about the neck of the user and the thyroid shield portion covers the thyroid of the user.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view showing a conventional soft collar;

FIG. 2 is a schematic view showing the thyroid adjacent other anatomical structures;

FIG. 3 is a schematic view showing a conventional thyroid shield;

FIGS. 4-6 are front, back and side views, respectively, of a novel radiation-protective neck support formed in accordance with the present invention;

FIGS. 7-9 are front, back and side views, respectively, showing the novel radiation-protective neck support of FIGS. 4-6 being worn by a user;

FIG. 10 is a side view showing the novel radiation-protective neck support of FIGS. 4-9, wherein the user is tilting their head downward and the invention is adequately flexible to accommodate this type of motion, which is frequently necessary during the performance of surgical procedures; and

FIG. 11 is a front view showing another novel radiation-protective neck support formed in accordance with the present invention.

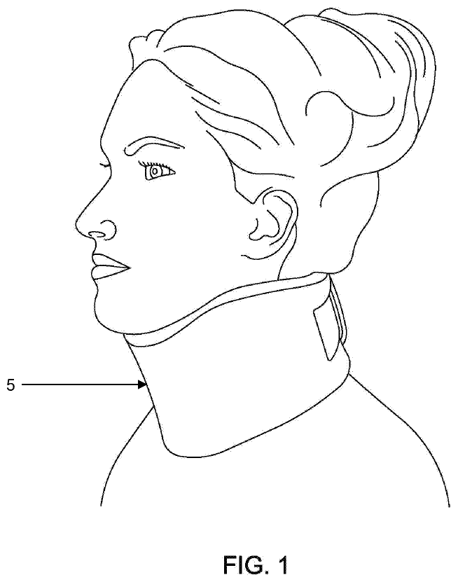

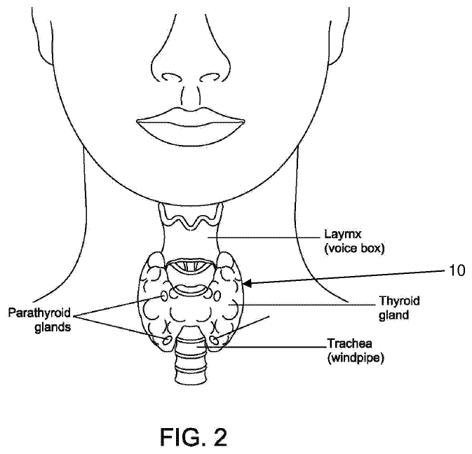

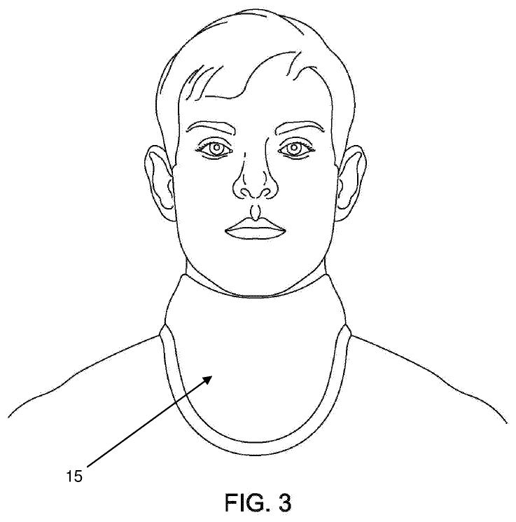

### DETAILED DESCRIPTION OF THE PREFERRED EMBODIMENTS

The present invention comprises the provision and use of a novel neck support with radiation protection.

More particularly, the present invention comprises the provision and use of a novel radiation-protective neck support which simultaneously provides both cervical support and radiation protection for the neck and thyroid of the user.

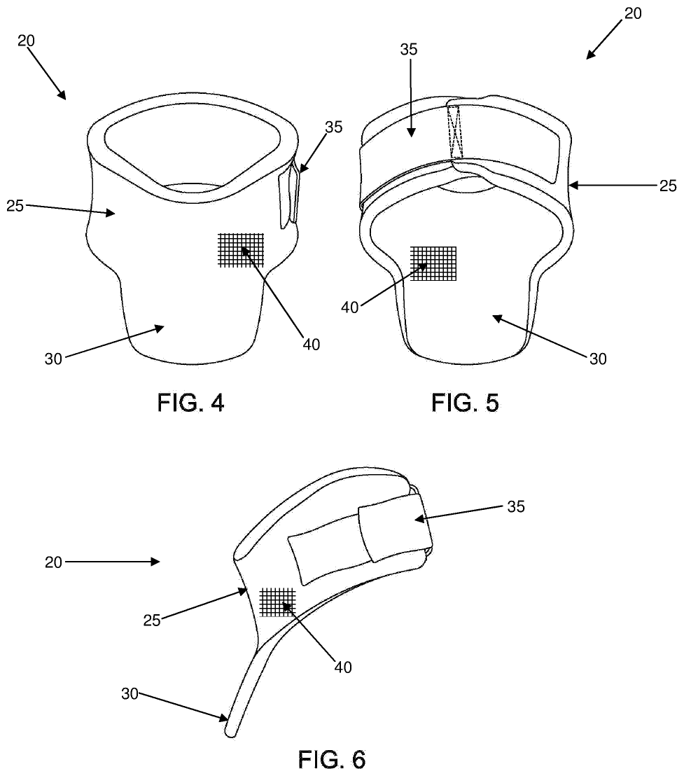

Looking now at FIGS. 4-6, there is shown a novel radiation-protective neck support 20 formed in accordance with the present invention. Radiation-protective neck support 20 generally comprises (i) a soft collar portion 25 which comprises a soft, flexible, supportive material structure (such as a supportive foam or a supportive gel pad or other supportive material with similar properties) and is “leaded” (or otherwise shielded against radiation), and (ii) a thyroid shield portion 30 which descends from soft collar portion 25 and is “leaded” (or otherwise shielded against radiation). Thyroid shield portion 30 can descend as a “flap” from soft collar portion 25 or it can attach in other ways to achieve the protection desired for the thyroid.

Soft collar portion 25 comprises a soft, flexible, supportive material structure (such as a supportive foam or a supportive gel pad or other supportive material with similar properties) which is capable of providing anatomical support and can “bend” so as to accommodate a range of user movements when it is fitted about the neck of a user, and lead or other suitable radiation-resistant material(s) (such as tungsten, barium, or other similar barrier materials) which provide(s) radiation protection to the underlying anatomy of a user. By way of example but not limitation, soft collar portion 25 may comprise a soft, flexible, supportive material structure (such as a supportive foam or a supportive gel pad or other supportive material with similar properties) which comprises a radiation-resistant coating (which may be applied to the outside or within the supportive material) which provides radiation protection to the underlying anatomy of a user without significantly stiffening soft collar portion 25 in the regions adjacent to the neck of the user to facilitate the movement necessary for procedure performance.

By way of further example but not limitation, soft collar portion 25 may comprise a supportive foam structure which comprises an outer coating of a radiation-resistant material(s) (such as lead, tungsten, barium, or other similar barrier materials) which provides radiation protection to the underlying anatomy of a user without significantly stiffening soft collar portion 25 in the regions adjacent to the neck of the user.

Alternatively, soft collar portion 25 may comprise a supportive foam structure which has a radiation-resistant material(s) (such as lead, tungsten, barium, or other similar barrier materials) dispersed throughout the foam structure which provides radiation protection to the underlying anatomy of a user without significantly stiffening soft collar portion 25 in the regions adjacent to the neck of the user.

Additionally, soft collar portion 25 may comprise a supportive foam structure which has a distinct layer or layers of radiation-resistant material(s) (such as lead, tungsten, barium, or other similar barrier materials) “sandwiched” within the foam structure (or other supportive structure) which provides radiation protection to the underlying anatomy of a user without significantly stiffening soft collar portion 25 in the regions adjacent to the neck of the user.

Soft collar portion 25 further comprises a closure device 35, e.g., a strap incorporating a hook-and-loop fastener, buttons, snaps, a conventional clasp, or other design, for holding radiation-protective neck support 20 in position around the neck of a user. It should be appreciated that closure device 35 may be adjustable so as to accommodate different neck sizes.

Additionally, radiation-protective neck support 20 comprises thyroid shield portion 30 which extends (e.g., in a flap-like manner) from the bottom of soft collar portion 25 so as to extend over the thyroid of the user. The junction between soft collar portion 25 and thyroid shield portion 30 is flexible, thus, thyroid shield portion 30 can “bend” relative to soft collar portion 25 and soft collar portion 25 can “bend” relative to the head and neck of a user so as to accommodate a range of user movements. Note that, if desired, thyroid shield portion 30 may also be flexible. Thyroid shield portion 30 comprises lead or other suitable material(s) (e.g., tungsten, barium, etc.) which provide(s) radiation protection to the underlying anatomy of the user. By way of example but not limitation, thyroid shield portion 30 comprises a layer of a radiation-resistant material(s) (such as lead, tungsten, barium, or other similar barrier materials) which provides radiation protection to the underlying anatomy of a user without significantly stiffening thyroid shield portion 30 in the regions adjacent to the anatomy of the user. By way of further example but not limitation, thyroid shield portion 30 may comprise radiation-resistant materials (such as lead, tungsten, barium, or other similar barrier materials) in particle or powder form carried by another structure (e.g., a “bean bag”-type of enclosure, a foam pad, etc.). It should be appreciated that thyroid shield portion 30 may comprise any radiation-resistant material suitable for use in protective equipment. It should also be appreciated that thyroid shield portion 30 is relatively light-weight (e.g., approximately 1 lb or less).

It should be appreciated that the radiation-shielding elements provided in soft collar portion 25 may be of the same type, material, form, thickness, weight, etc. as the radiation-shielding elements provided in thyroid shield portion 30. Or the radiation-shielding elements provided in soft collar portion 25 may be of a different type, material, form, thickness, weight, etc. as the radiation-shielding elements provided in thyroid shield portion 30. Stated another way, the radiation-shielding elements provided in soft collar portion 25 may be similar to, or different from, the radiation-shielding elements provided in thyroid shield portion 30 with regard to type, material, thickness, weight, etc.

In one preferred form of the invention, soft collar portion 25 and thyroid shield portion 30 are formed as an integrated structure, with soft collar portion 25 and thyroid shield portion 30 being “bendable” so as to accommodate a range of user movements.

In one preferred form of the invention, the integrated structure of soft collar portion 25 and thyroid shield portion 30 (i.e., the complete radiation-protective neck support 20) are covered by a single fabric covering 40 which has a soft and comfortable texture. Fabric covering 40 can be disposable or secured to radiation-protective neck support 20.

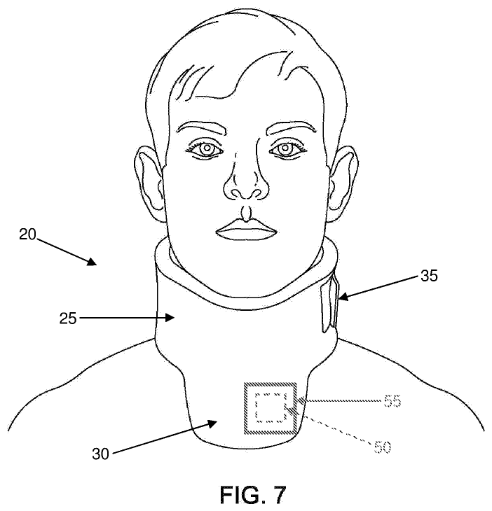

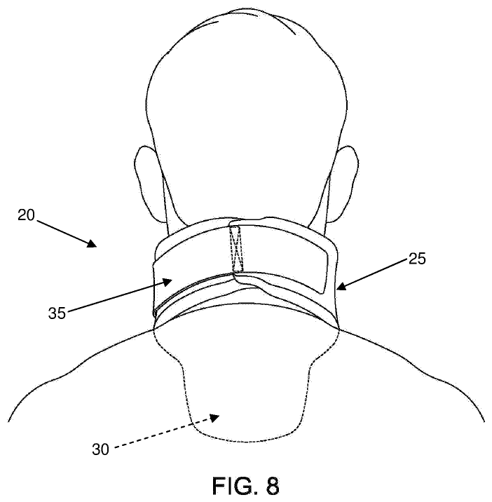

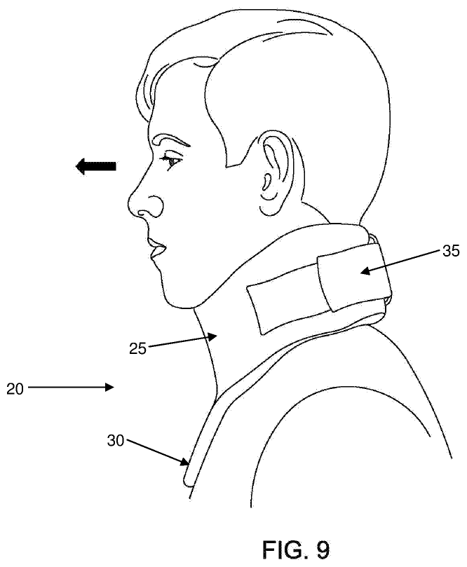

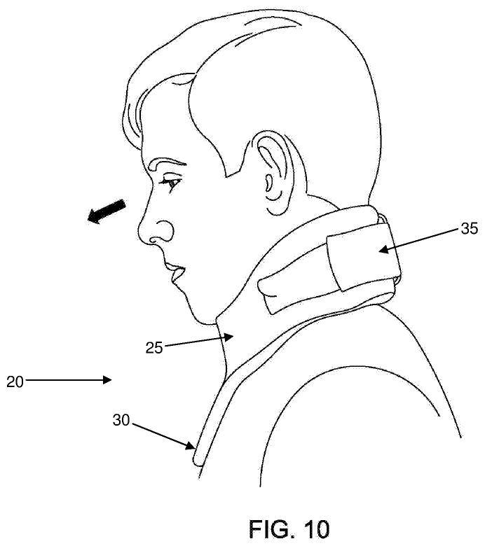

During surgery, and looking next at FIGS. 7-10, radiation-protective neck support 20 is worn by the user, with soft collar portion 25 surrounding the neck of the user, thereby providing anatomical support to the neck of the user and also providing radiation protection for the neck of the user, and with thyroid shield portion 30 descending as a “flap” from soft collar portion 25 so as to overlay the thyroid of the user and thereby providing radiation protection for the thyroid of the user. Soft collar portion 25 and thyroid shield portion 30 can “bend” so as to accommodate a range of user movements.

Radiation-protective neck support 20 is intended to be put on prior to scrubbing, in a manner similar to how conventional radiation-resistant protective equipment is dawned prior to scrubbing. Soft collar portion 25 of radiation-protective neck support 20 supports the anatomy of the user from the shoulders to the mandible. The fact that collar portion 25 is soft allows for easy neck movement by the user, as compared to the restrictive constraint associated with rigid collars; however, as the user maintains a bent-over posture throughout a surgical procedure, the load on their posterior neck muscles is relieved by the inherent chin support conferred by soft collar portion 25 of radiation-protective neck support 20.

Thus it will be seen that with the present invention, the user has relief from the neck-related pain and problems seen amongst surgeons from remaining in ergonomically-disadvantageous positions for prolonged periods of time during surgical procedures, and the user has protection against radiation exposure to the neck and the thyroid of the user.

### Additional Constructions

It is possible to modify radiation-protective neck support 20 while remaining within the scope of the present invention.

By way of example but not limitation, fabric covering 40 may be constructed so that it can be periodically removed from radiation-protective neck support 20, e.g., for laundering.

By way of further example but not limitation, radiation-protective neck support 20 may be constructed so that thyroid shield portion 30 is formed as a separate unit from soft collar portion 25, with thyroid shield portion 30 being united with soft collar portion 25 at the time of use. There are different ways to unite them, including but not limited to use of a cloth, plastic or mechanical hinge or pouch arrangement, between these two parts or between coverings. In this form of the invention, fabric covering 40 can be applied over the unified assembly of soft collar portion 20 and thyroid shield portion 30 after the two portions have been united, or separate fabric coverings 40 may be provided for each of unified soft collar portion 20 and thyroid shield portion 30. It should be appreciated that with this construction of radiation-protective neck support 20, soft collar portion 25 can be periodically replaced when necessary, e.g., when it becomes “dirty” or worn.

Furthermore, radiation-protective neck support 20 may be constructed so that only thyroid portion 30 includes radiation protection, e.g., soft collar portion 25 could be lead-free, tungsten-free, barium-free, etc.

By way of further example but not limitation, and looking now at FIG. 7, a pouch 50 may be constructed on the outer surface of soft collar portion 25 or thyroid shield portion 30 so as to allow for placement of a radiation dosimeter 55, which may be used to record the amount of radiation exposure in the region of application. Such a pouch can also be used with a cover placed over radiation-protective neck support 20.

Additionally, radiation-protective neck support 20 may be constructed so as to be disposable, i.e., it may be provided in the form of a single-use radiation-protective neck support which may be disposed of after a user has completed a medical procedure.

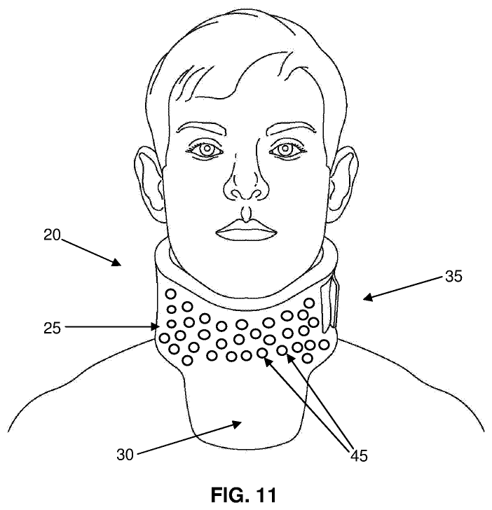

By way of further example but not limitation, and looking now at FIG. 11, soft collar portion 25 may comprise a plurality of small holes 45, preferably disposed in a staggered pattern, so as to improve the flexibility of soft collar portion 25. Holes 25 are sized so as to be large enough to increase the flexibility of soft collar portion 25 without significantly detracting from the radiation protection provided by soft collar portion 25. Additionally, the plurality of small holes 45 in soft collar portion 25 may improve the breathability of radiation-protective neck support 20, thus providing greater comfort to the user.

### Modifications of the Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. A radiation-protective neck support comprising:
a soft collar portion comprising an anatomical support structure for the neck of a user, and radiation shielding for protection of a user's anatomy underlying the soft collar portion; and
a thyroid shield portion comprising radiation shielding for providing radiation protection to the user's anatomy underlying the thyroid shield portion, wherein the thyroid shield portion descends from the soft collar portion and is sized to cover the thyroid of a user when the soft collar portion is fitted about the neck of a user.

2. A radiation-protective neck support according to claim 1 wherein the thyroid shield portion is flexibly secured to the soft collar portion so as to accommodate a range of user movements.

3. A radiation-protective neck support according to claim 1 wherein the soft collar portion and the thyroid shield portion are formed as an integrated structure.

4. A radiation-protective neck support according to claim 1 further comprising a single fabric covering.

5. A radiation-protective neck support according to claim 1 wherein the radiation shielding of the soft collar portion comprises a material selected from the group consisting of lead, tungsten, barium or other radiation-resistant material suitable for use in protective equipment.

6. A radiation-protective neck support according to claim 1 wherein the radiation shielding of the soft collar portion comprises an outer coating of a radiation-resistant powder secured to a supportive foam structure.

7. A radiation-protective neck support according to claim 6 wherein the radiation-resistant powder is secured to the supportive foam structure so as to provide radiation protection to the anatomy of a user underlying the soft collar portion without significantly stiffening the supportive foam structure.

8. A radiation-protective neck support according to claim 1 wherein the radiation shielding of the thyroid shield portion comprises a material selected from the group consisting of lead, tungsten, barium or other radiation-resistant material suitable for use in protective equipment.

9. A radiation-protective neck support according to claim 1 wherein the soft collar portion comprises a plurality of holes.

10. A radiation-protective neck support according to claim 9 wherein the plurality of holes are configured in a staggered pattern.

11. A method for providing neck support and radiation protection for a user, the method comprising:
providing a radiation-protective neck support, the radiation-protective neck support comprising:
a soft collar portion comprising an anatomical support structure for the neck of a user, and radiation shielding for protection of a user's anatomy underlying the soft collar portion; and
a thyroid shield portion comprising radiation shielding for providing radiation protection to the user's anatomy underlying the thyroid shield portion, wherein the thyroid shield portion descends from the soft collar portion and is sized to cover the thyroid of a user when the soft collar portion is fitted about the neck of a user; and
positioning the radiation-protective neck support on a user so that the soft collar portion is fitted about the neck of the user and the thyroid shield portion covers the thyroid of the user.

12. A method according to claim 11 wherein the thyroid shield portion is flexibly secured to the soft collar portion so as to accommodate a range of user movements.

13. A method according to claim 11 wherein the soft collar portion and the thyroid shield portion are formed as an integrated structure.

14. A method according to claim 11 wherein the radiation-protective neck support further comprises a single fabric covering.

15. A method according to claim 11 wherein the radiation shielding of the soft collar portion comprises a material selected from the group consisting of lead, tungsten, barium or other radiation-resistant material suitable for use in protective equipment.

16. A method according to claim 11 wherein the radiation shielding of the soft collar portion comprises an outer coating of a radiation-resistant powder secured to a supportive foam structure.

17. A method according to claim 16 wherein the radiation-resistant powder is secured to the supportive foam structure so as to provide radiation protection to the anatomy of a user underlying the soft collar portion without significantly stiffening the supportive foam structure.

18. A method according to claim 11 wherein the radiation shielding of the thyroid shield portion comprises a material selected from the group consisting of lead, tungsten, barium or other radiation-resistant material suitable for use in protective equipment.

19. A method according to claim 11 wherein the soft collar portion comprises a plurality of holes.

20. A method according to claim 19 wherein the plurality of holes are configured in a staggered pattern.
